(19) United States
(12) Plant Patent Application Publication
(10) Pub. No.: US 2014/0189917 P1
(43) Pub. Date: Jul. 3, 2014

(54) Streptocarpus plant named 'SLSDB17-0'

(50) ;

(71) Applicant: Lynne Dibley, Ruthin (GB)

(72) Inventor: Lynne Dibley, Ruthin (GB)

(21) Appl. No.: 13/694,735

(22) Filed: Dec. 27, 2012

(51) Int. Cl. A01H 5/00

(52) U.S. Cl. PLT/333

(57) ABSTRACT

A new and distinct Streptocarpus cultivar named ‘SLSDB17-0’ is disclosed, characterized by large, unusually marked flowers with white upper lobes and heavily veined lower lobes. Plants are highly floriferous. The new variety is a Streptocarpus, suitable as an outdoor garden or container plant.

### LATIN NAME OF THE GENUS AND SPECIES

Streptocarpus hybrid

### VARIETY DENOMINATION

‘SLSDB17-0’

The new Streptocarpus cultivar is the product of a planned breeding program by the inventor Lynne Dibley, a citizen of United Kingdom. The breeding program was carried out at a commercial nursery in Ruthin, United Kingdom. The objective of the breeding program was to develop new Streptocarpus varieties with good characteristics for ornamental purposes.

The new variety resulted from the crossing of the seed parent, an unnamed, unpatented proprietary Streptocarpus seedling with the pollen parent, an unnamed, unpatented proprietary Streptocarpus seedling in 2010. The new variety was discovered in 2011 by the inventor in a group of seedlings resulting from the 2010 crossing, at a commercial nursery in Ruthin, United Kingdom.

Asexual reproduction of the new cultivar ‘SLSDB17-0’ by vegetative cuttings was first performed at a commercial greenhouse in Ruthin, United Kingdom in 2011. Subsequently at least three generations have been produced from vegetative cuttings, and have shown that the unique features of this cultivar are stable and reproduced true to type.

### SUMMARY OF THE INVENTION

The cultivar ‘SLSDB17-0’ has not observed under all possible environmental conditions. The phenotype may vary somewhat with variations in environment such as temperature, day length and light intensity, without however, any variance in genotype.

The following traits have been repeatedly observed and are determined to be the unique characteristics of ‘SLSDB17-0.’ These characteristics in combination distinguish ‘SLSDB17-0’ as a new and distinct Streptocarpus cultivar:

1. Large flowers size.

2. Heavily veined on lower 3 floral lobes.

3. Highly floriferous plants.

4. White upper lobes.

5. Unusual flower markings.

### COMPARISON TO THE PARENT VARIETIES

Plants of the new cultivar ‘SLSDB17-0’ are similar in most horticultural characteristics to plants of the unnamed, unpatented proprietary seed parent. However, the new variety has a larger overall flower and smaller leaf size than the seed parent. Additionally, plants of ‘SLSDB17-0’ produce different colored flowers, with different markings, as well as shorter peduncles than the seed parent.

Plants of the new cultivar ‘SLSDB17-0’ are similar in most horticultural characteristics to plants of the unnamed, unpatented proprietary pollen parent. However, the new variety has a larger flower and smaller leaf size than the pollen parent. Additionally, plants of ‘SLSDB17-0’ produce flowers with different colored lobes and markings than the pollen parent.

### COMPARISON TO COMMERCIAL VARIETY

The new cultivar can be compared to the commercial variety ‘Harlequin Blue’, unpatented. Plants of the two cultivars are similar in most horticultural characteristics, however, plants of ‘SLSDB17-0’ produce more leaves and larger leaves as well as flowering earlier. Additionally, plants of ‘SLSDB17-0’ produce larger flowers with different floral lobe coloring.

The new cultivar can be compared to the commercial variety ‘Bethan’, unpatented. Plants of the two cultivars are similar in most horticultural characteristics, however, plants of ‘SLSDB17-0’ produce larger flowers with different lobe coloring. Additionally, plants of ‘SLSDB17-0’ produce more leaves of larger size.

### BRIEF DESCRIPTION OF THE PHOTOGRAPHS

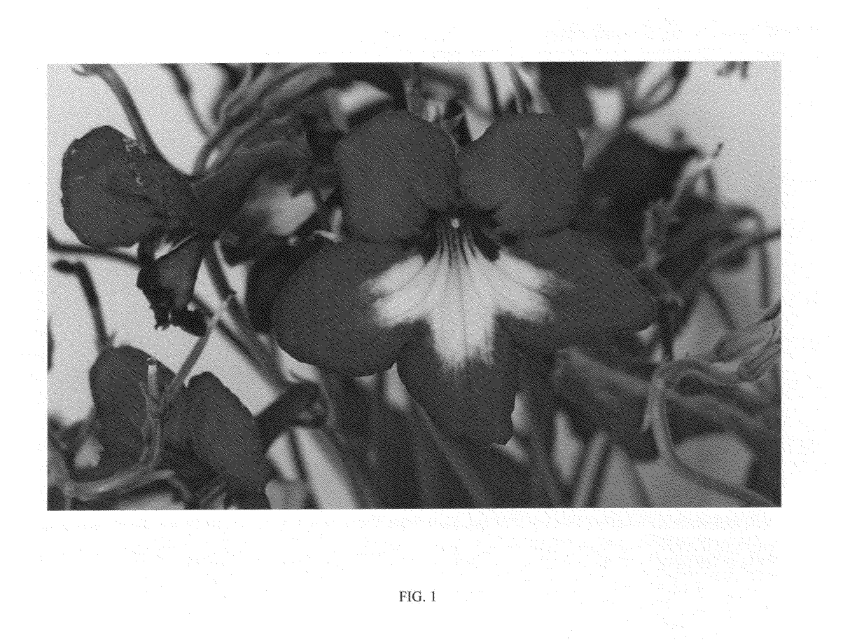

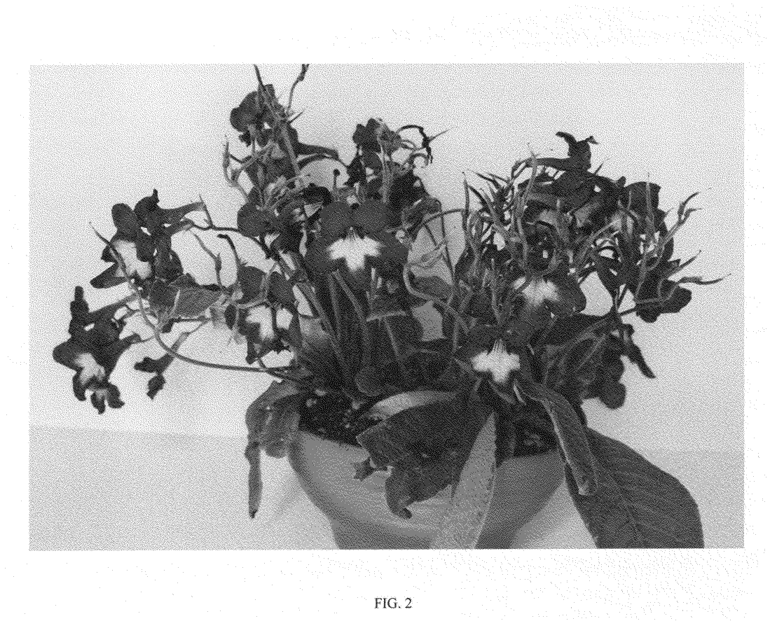

The accompanying photograph in FIG. 1 illustrates in full color a typical flower of ‘SLSDB17-0’. FIG. 2 illustrates a typical plant of ‘SLSDB17-0’ grown in a greenhouse, in an 8 inch commercial pot. Age of the plant photographed is approximately 30 weeks from a rooted cutting. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart, 2001 except where general terms of ordinary dictionary significance are used. The following observations and measurement describe ‘SLSDB17-0’ plants grown in a commercial greenhouse in Oxnard, Calif. The growing temperature ranged from approximately 12° C. to 30° C. The greenhouse is subject to partial shade. General light conditions are bright, normal sunlight with some shade during the brightest part of the day. Measurements and numerical values represent averages of typical plant types.
Botanical classification: Streptocarpus hybrid ‘SLSDB17-0’
Propagation:
   Time to initiate roots.—About 20 to 25 days at approximately 21 to 25° C.
   Root description.—Fine, fibrous.
Plant:
   Growth habit.—Upright.
   Age of the plant described.—Approximately 30 weeks from a well rooted cutting.
   Pot size of plant described.—8 inch pot.
   Height.—Approximately 13 cm from soil line to top of foliar plane. Approximately 23 cm to top of highest flower.
   Plant spread.—Approximately 35 cm at widest point.
   Growth rate.—Moderate.
   Branching characteristics.—Basal rosettes of foliate, no stem, no branches.
Foliage:
   Leaf.—Arrangement: Rosette Quantity: Average 12 to 55 leaves. Average Length: Approximately 17 cm. Average Width: Approximately 6 cm. Shape of blade: Lanceolate Apex: Acute Base: Attenuate Margin: Crenate Appearance of top surface: Matte Appearance of top surface: Matte Pubescence: Very short, less than 1 mm hairs, densely covering top and bottom leaf surfaces. Aspect: Somewhat undulating, overall leaf reflexes downward in older foliage. Color: Young foliage upper side: Near RHS Green 137C Young foliage under side: Near RHS Green 138B Mature foliage upper side: Near RHS Green 137B Mature foliage under side: Near RHS Green 138B Venation: Type: Pinnate. Venation color upper side: Near RHS Green 143B Venation color under side: Near RHS Yellow-Green 145B Petiole: Not present, leaves sessile.
Flower:
   Natural flowering season.—Continuous.
   Inflorescence and flower type and habit.—Racemes of 2 to 4 asymmetrical funnel shaped flowers.
   Inflorescence longevity on plant.—At least 2 weeks before any significant change is observed.
   Approximate quantity of flowers per plant.—Approximately 45 mature and immature flowers per plant.
   Flowers persistent.—
   Inflorescence.—Diameter of entire Inflorescence: Approximately 13.5 cm. Depth of Inflorescence: Approximately 8.0 cm.
   Individual flower bud.—Shape: Tubular, with spherical apex Length: Approximately 4.0 cm. Diameter: Approximately 1.9 cm Color: Near RHS Purple N79D, apex Purple N79A.
   Flowers.—
   Flower length.—6.5 cm.
   Flower width.—7.0 cm. Upper petals (upper lip): Number: 2, fused together at base. Length: 3.0 cm Width: 2.5 cm Shape: Obicular Apex: Obtuse, ruffled. Margin: Shallow crenate Texture: Velvety, all surfaces. Pubescent outer surface. Color: When opening: Inner surface: RHS Purple-Violet N81B Outer surface: RHS Purple N78A Fully opened: Inner surface: RHS Purple-Violet N81A, base flushed Purple N79A Outer surface: RHS Purple N78A Aging: Inner surface: RHS Violet 83A, base Purple N79A Outer surface: RHS Purple 77A Lower petals (lower lip): Quantity: 3 Length: 3.0 cm Width: 2.5 cm Shape: Orbicular, fused at base. Apex shape: Obtuse Margin: Shallow crenate, undulating Texture: Velvety, all surfaces. Pubescent outer surface. Color: When opening: Inner surface: RHS Purple-Violet N81A, base Yellow-White 158D and Yellow 10D. Outer surface: RHS Purple-Violet N81A Fully opened: Inner surface: RHS Purple-Violet N81A, base Yellow-White 158D. Outer surface: RHS Purple-Violet N81A Aging: Inner surface: RHS Violet 83A, base Yellow-White 158B. Outer surface: RHS Purple N79C Throat: Length: 3.0 cm Width: 1 cm, at widest point Texture: glabrous, shiny inner surface. Outer surface pubescent. Color: When opening: Inner surface: RHS Purple-Violet N81 with stripes Yellow-White 158D and Yellow 10D. Outer surface: RHS Purple N79C Fully opened: Inner surface: RHS Purple-Violet N81 with stripes Yellow-White 158D and Yellow 10D. Outer surface: RHS Purple N79C with stripes Purple N78C Aging: Inner surface: RHS Purple-Violet N81 with stripes Yellow-White 158D and Yellow 10D. Outer surface: RHS Purple N79C with stripes Purple N78C.
   Sepals.—Quantity: Average 5 Shape: Narrow deltate, base fused Length: Approximately 0.6 cm Width: Approximately 0.2 cm Apex: Acute Base: Fused Margin: Entire Texture: Highly pubescent, all surfaces. Color: Upper Surface: Near RHS Green 137A. Lower Surface: Near RHS Green 137A.
   Peduncle.—Length: Longest average 16 cm. Shortest average 10 cm Diameter: Approximately 0.4 cm. Texture: Densely pubescent. Color: Near RHS Yellow-Green 144B Orientation: Approximately 90 to 75 degree angle from rosette undulating.
   Pedicel.—Length: Average range 0.8 cm to 1.5 cm Diameter: Approximately 0.3 cm. Texture: Soft, dense pubescence. Color: Near RHS Yellow-Green 144B, flushed with Purple N77A Orientation: Approximately 30 degree angle from peduncle. Straight, or slightly outwardly curved.
   Fragrance.—None.
Reproductive organs:
   Stamens.—Number: 2 Filament length: Approximately 0.7 cm, unfused section.
   Anthers.—Length: Approximately 0.2 cm Shape: Bi-lobed Color: Near RHS White N155C Pollen: Not observed.
   Pistil.—Number: 1 Length: Approximately 2.5 cm Style: Length: Approximately 2.0 cm. Color: Near RHS Greyed-Purple N187C from base to approximately 50% of length. Upper 50% near White N155A. Stigma: Shape: Bifurcate Color: Near RHS White N155C.
Other characteristics:
   Seeds and fruits.—Not observed.
   Disease/pest resistance.—Neither resistant nor susceptible to normal diseases and pests of Streptocarpus.
   Temperature tolerance.—USDA zones 9-11.

What is claimed is:

1. A new and distinct cultivar of Streptocarpus plant named ‘SLSDB17-0’ as herein illustrated and described.
